(19) United States
(12) Patent Application Publication
(10) Pub. No.: US 2019/0321283 A1
(43) Pub. Date: Oct. 24, 2019

(54) COMPOSITION AND METHOD FOR PREPARING A CUSTOMIZED PERSONAL CARE PRODUCT

(71) Applicant: Jelena Hacaturjanca, London (GB)

(72) Inventor: Jelena Hacaturjanca, London (GB)

(21) Appl. No.: 16/135,587

(22) Filed: Sep. 19, 2018

(30) Foreign Application Priority Data
Apr. 20, 2018 (GB) 1806423.8

(51) Int. Cl. A61K 8/92; A61K 8/9789; A61K 8/42; A61K 8/365; A61K 8/67; A61K 8/368; A61K 8/34; A61K 8/65; A61K 8/37; A61K 8/60; A61Q 7/00; A61Q 5/00

(52) U.S. Cl. CPC A61K 8/922; A61K 8/9789; A61K 8/42; A61K 8/365; A61K 8/678; A61K 8/368; A61Q 19/00; A61K 8/65; A61K 8/37; A61K 8/602; A61Q 7/00; A61Q 5/006; A61Q 5/008; A61K 8/345

(57) ABSTRACT

The present invention provides a composition and method for preparing a customized personal care product. The composition comprising a base composition and at least one additive compositions. The base composition is at least one or combination of organic or inorganic ingredients and organic origin ingredients, and the additive compositions include at least one hair care based additive compositions. at least one customized additive of predetermined composition from an ampoule is added to the base composition. The base composition and at least one customized additive compositions are mixed to form a customized personal care composition. The personal care product could be a health care product, beauty product, face serum product, body lotion product, hair care product, or any other product.

### CROSS REFERENCE TO RELATED APPLICATION

The present application claims the benefit of UK Patent Application GB1806423.8 for “Composition and Method for Preparing a Customized Personal Care Product”, filed Apr. 20, 2018, the contents of which is hereby incorporated by reference.

### BACKGROUND OF THE INVENTION

### A. Technical Field

The present invention generally relates to personal care product, and more specifically relates to a composition and method for preparing a customized personal care product.

### B. Description of Related Art

The trend of allowing user to choose from various types of differentiated composition for a same product is known. For instance, in shampoo, the consumer can not only purchase a regular shampoo for washing hair, but Clarifying shampoo, Volumizing shampoo, Oily hair shampoo, Damaged hair shampoo, Colored hair shampoo, Medicated shampoo and Swimmer's shampoo. similarly, each personal care products such as face serum, body cream, health products have wide choice for the user to choose for each personal care product.

Even with the wide choice of products, the customer could not get a product that is specifically tailored to them. Also, providing a variety of choices for a particular product creates manufacturing and inventory issues to the manufacturers. For example, where a product has a limited shelf-life, manufacturing of a plurality of different composition without certain about the individual customer need, often leads to increased waste due to expiration of unused product. However, the ability to customize or personalize consumer products has become increasingly popular and desirable in recent years. Consumers have shown an interest in the ability to create products that reflect their personal interests and tastes.

Therefore, a need exists for techniques to allow consumers to personalize products for their specific needs. a further need exist for a composition and method to a enables a user to customize a personal care product.

### SUMMARY OF THE INVENTION

The present invention provides a composition and method for preparing a customized personal care product. The composition comprising a base composition and at least one additive compositions. The base composition is at least one or combination of organic or inorganic ingredients and organic origin ingredients, and the additive compositions include at least one hair care based additive compositions. The base composition includes sodium coco-sulphate, cocamidopropyl betaine, decyl glucoside, glycerin, caprylyl/capryl glucoside, lauryl glucoside, aloe barbadensis leaf juice powder, glyceryl oleate, coco-glucoside, citric acid, levulinic acid, sodium levulinate, sodium phytate, sodium benzoate and water. The personal care product could be a health care product, beauty product, face serum product, body lotion product, hair care product, or any other product.

According to the present invention, the hair care based additive composition for hair loss include, (a) 40% of Wu Zhu extract (propanediol and Evodia rutaecarpa fruit extract), (b) 5% of panthenol, (c) 1% of caffeine, (d) 40% of green tea extract, (e) 0.5% of lactic acid, (f) 2% of vitamin E, (g) 1% of tea tree oil, (h) 1% of rosemary oil, (i) 0.5% of sodium benzoate, and (j) 9% of PEG-40 hydrogenated castor oil.

According to the present invention, the hair care based additive composition for hair root strengthening include, (a) 3% of powdered Aloe vera extract, (b) 41.2% of water, (c) 20% of horse tail tincture, (d) 15% of Nettle tincture, (e) 5% of Pea protein, (f) 5% of Dandelion extract, (g) 5% of Panthenol, (h) 5% of Glycerin, and (i) 0.8% of Preservative 12 (Phenoxyethanol, Ethylhexylglycerin).

According to the present invention, the hair care based additive composition for voluminous hair include, (a) 80% of Phytokeratin, (b) 10% of Wheat Protein, (c) 5% of Pseudo Collagen, (d) 4.2% of Silk amino acids, and (e) 0.8% of Preservative 12 (Phenoxyethanol, Ethylhexylglycerin).

According to the present invention, the hair care based additive composition for dry brittle hair include, (a) 70% of Caprylic/Capric triglicerides, (b) 5% of Prickly Pear Seed oil, (c) 10% of Bao bab oil, (d) 10% of Oat oil, (e) 4% of Argan oil and (f) 1% of Vitamin E.

According to the present invention, the hair care based additive composition for dull hair include, (a) 70% of Caprylic/Capric triglicerides, (b) 5% of Avocado oil, (c) 14% of Jojoba oil, (d) 10% of Camellia seed oil, (e) 2% of Castor oil, (f) 2% of Rice bran oil, (g) 2% of Safflower Oil, and (h) 1% of Vitamin E.

According to the present invention, the hair care based additive composition for frizzy hair include, (a) 43.2% of water, (b) 6% of Glycerin, (c) 5% of Pantenol, (d) 15% of Phytokeratin, (e) 5% of Broccoli oil, (f) 5% of Polyquaternium-7, (g) 10% of PEG-40 hydrogenated castor oil, and (h) 0.8% of Preservative 12 (Phenoxyethanol, Ethylhexylglycerin).

According to the present invention, the hair care based additive composition for dandruff or psoriasis treatment include, (a) 1.5% of Salicylic acid, (b) 72.5% of Propylen glycol, (c) 1% of Bisabolol, (d) 10% of Tea tree oil, (e) 10% of rosemary oil, and (d) 5% of cosmetic prebiotic (inulin, Aqua, Alpha-glucan oligosaccharide).

According to the present invention, the hair care based additive composition for oily scalp include, (a) 57.2% of Rose floral water, (b) 10% of Cosmetic Prebiotic (inulin, Aqua, Alpha-glucan oligosaccharide), (c) 10% of Alpine extract, (d) 2% of Tea tree oil, (e) 3% of Clary sage oil, (f) 2% of Cedar wood oil, (g) 15% of PEG hydrogenated castor oil, and (h) 0.8% of Preservative 12 (Phenoxyethanol, Ethylhexylglycerin).

According to the present invention, the hair care based additive composition for replumping hair filler include, (a) 80% of Phytokeratin, (b) 10% of Panthenol, (c) 10% of Pseudo Collagen, and (d) 10% of Preservative 12 (Phenoxyethanol, Ethylhexylglycerin).

According to the present invention, a method for preparing a customized personal care composition is disclosed. at one step, a base composition is prepared. At another step, at least one customized additive of predetermined composition from an ampoule is added to the base composition. At another step, the base composition and at least one customized additive compositions are mixed to form a customized personal care composition. The base composition is a combination of organic or inorganic ingredients and organic origin ingredients, and the additive compositions include at least one hair care based additive compositions.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF DRAWINGS

The embodiments herein will be better understood from the following detailed description with reference to the drawings, in which:

FIG. 1 is a flowchart exemplarily illustrating a method for preparing a customized personal care composition in an embodiment of the present invention.

### DETAILED DESCRIPTION OF EMBODIMENTS

A description of embodiments of the present invention will now be given with reference to the Figures. It is expected that the present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

The present invention provides a composition and method for preparing a customized personal care product. In one embodiment, the personal care product could be a face serum, body lotion, health products, hair care product beauty product, or any other product. The composition comprising a base composition and at least one additive compositions. The base composition is at least one or combination of organic or inorganic ingredients and organic origin ingredients, and the additive compositions include at least one hair care based additive compositions. In one embodiment, the base composition includes sodium coco-sulphate, cocamidopropyl betaine, decyl glucoside, glycerin, caprylyl/capryl glucoside, lauryl glucoside, aloe barbadensis leaf juice powder, glyceryl oleate, coco-glucoside, citric acid, levulinic acid, sodium levulinate, sodium phytate, sodium benzoate and water.

In on embodiment, the base composition includes 5.0 to 0.10.0 percent of sodium coco-sulphate, 1.0 to 0.5.0 percent of cocamidopropyl betaine, 1.0 to 0.5.0 percent of decyl glucoside, 1.0 to 0.5.0 percent of glycerin, 1.0 to 0.5.0 percent of caprylyl/capryl glucoside, 0.1 to 0.1 percent of lauryl glucoside, 0.1 to 0.1 percent of aloe barbadensis leaf juice powder, 0.1 to 0.1 percent of glyceryl oleate, 0.1 to 0.1 percent of coco-glucoside, 0.1 to 0.1 percent of citric acid, 0.1 to 0.1 percent of levulinic acid, 0.1 to 0.1 percent of sodium levulinate, 0.1 to 0.1 percent of sodium phytate, 0.1 to 0.1 percent of sodium benzoate and 75.0 to 100.0 percent of water.

In one embodiment, the hair care based additive composition for hair loss include, (a) 40% of Wu Zhu extract (propanediol and Evodia rutaecarpa fruit extract), (b) 5% of panthenol, (c) 1% of caffeine, (d) 40% of green tea extract, (e) 0.5% of lactic acid, (f) 2% of vitamin E, (g) 1% of tea tree oil, (h) 1% of rosemary oil, (i) 0.5% of sodium benzoate, and (j) 9% of PEG-40 hydrogenated castor oil.

In one embodiment, the hair care based additive composition for hair root strengthening include, (a) 3% of powdered Aloe vera extract, (b) 41.2% of water, (c) 20% of horse tail tincture, (d) 15% of Nettle tincture, (e) 5% of Pea protein, (f) 5% of Dandelion extract, (g) 5% of Panthenol, (h) 5% of Glycerin, and (i) 0.8% of Preservative 12 (Phenoxyethanol, Ethylhexylglycerin).

In one embodiment, the hair care based additive composition for voluminous hair include, (a) 80% of Phytokeratin, (b) 10% of Wheat Protein, (c) 5% of Pseudo Collagen, (d) 4.2% of Silk amino acids, and (e) 0.8% of Preservative 12 (Phenoxyethanol, Ethylhexylglycerin).

In one embodiment, the hair care based additive composition for dry brittle hair include, (a) 70% of Caprylic/Capric triglicerides, (b) 5% of Prickly Pear Seed oil, (c) 10% of Bao bab oil, (d) 10% of Oat oil, (e) 4% of Argan oil and (f) 1% of Vitamin E.

In one embodiment, the hair care based additive composition for dull hair include, (a) 70% of Caprylic/Capric triglicerides, (b) 5% of Avocado oil, (c) 14% of Jojoba oil, (d) 10% of Camellia seed oil, (e) 2% of Castor oil, (f) 2% of Rice bran oil, (g) 2% of Safflower Oil, and (h) 1% of Vitamin E.

In one embodiment, the hair care based additive composition for frizzy hair include, (a) 43.2% of water, (b) 6% of Glycerin, (c) 5% of Pantenol, (d) 15% of Phytokeratin, (e) 5% of Broccoli oil, (f) 5% of Polyquaternium-7, (g) 10% of PEG-40 hydrogenated castor oil, and (h) 0.8% of Preservative 12 (Phenoxyethanol, Ethylhexylglycerin).

In one embodiment, the hair care based additive composition for dandruff or psoriasis treatment include, (a) 1.5% of Salicylic acid, (b) 72.5% of Propylen glycol, (c) 1% of Bisabolol, (d) 10% of Tea tree oil, (e) 10% of rosemary oil, and (d) 5% of cosmetic prebiotic (inulin, Aqua, Alpha-glucan oligosaccharide).

In one embodiment, the hair care based additive composition for oily scalp include, (a) 57.2% of Rose floral water, (b) 10% of Cosmetic Prebiotic (inulin, Aqua, Alpha-glucan oligosaccharide), (c) 10% of Alpine extract, (d) 2% of Tea tree oil, (e) 3% of Clary sage oil, (f) 2% of Cedar wood oil, (g) 15% of PEG hydrogenated castor oil, and (h) 0.8% of Preservative 12 (Phenoxyethanol, Ethylhexylglycerin).

In one embodiment, the hair care based additive composition for replumping hair filler include, (a) 80% of Phytokeratin, (b) 10% of Panthenol, (c) 10% of Pseudo Collagen, and (d) 10% of Preservative 12 (Phenoxyethanol, Ethylhexylglycerin).

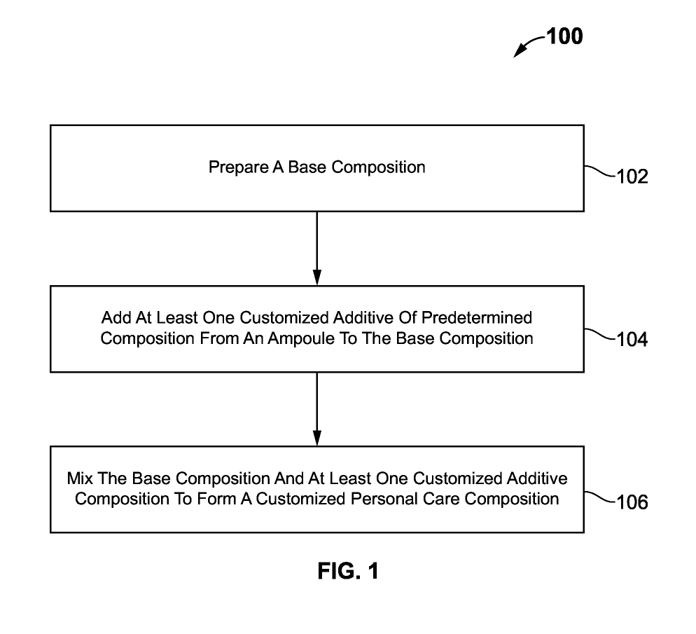

Referring to FIG. 1, a method 100 for preparing a customized personal care composition is disclosed in an embodiment of the present invention. At step 102, a base composition is prepared. At step 104, at least one customized additive of predetermined composition from an ampoule is added to the base composition. At step 106, the base composition and at least one customized additive compositions are mixed to form a customized personal care composition. The base composition is at least one or combination of organic or inorganic ingredients and organic origin ingredients, and the additive compositions include at least one hair care based additive compositions. Advantageously, the present allows consumer to personalize products for their specific needs.

The invention is further explained in the following examples, which however, are not to be construed to limit the scope of the invention.

### EXAMPLES

### Example—1

In an example, the hair care based additive composition for hair loss in weight percentage, grams per 5 ml ampules, weight percentage in 400 ml shampoo and weight percentage in 100 ml shampoo is disclosed in the following table.

| | | | % in | % in |
|---|---|---|---|---|
| | | g/5 ml | shampoo | shampoo |
| Ingredients | % | ampules | 400 ml | 100 ml |
| Wu Zhu (Propanediol, | 40 | 2 | 0.5 | 2 |
| Evodia rutaecarpa Fruit | | | | |
| Extract) | | | | |
| Panthenol | 5 | 0.25 | 0.0625 | 0.25 |
| Caffeine | 1 | 0.05 | 0.025 | 0.05 |
| Green tea extract | 40 | 2 | 0.5 | 2 |
| Lactic acid | 0.5 | 0.025 | 0.00625 | 0.025 |
| Vitamin E | 2 | 0.1 | 0.025 | 0.1 |
| Tea tree oil | 1 | 0.05 | 0.025 | 0.05 |
| Rosemary essential oil | 1 | 0.05 | 0.0125 | 0.05 |
| Sodium Benzoate | 0.5 | 0.025 | 0.00625 | 0.025 |
| Peg 40 hydrogenated castor | 9 | 0.45 | 0.11 | 0.45 |
| oil liquid | | | | |

### Example—2

In an example, the hair care based additive composition for hair root strengthening in weight percentage, grams per 5 ml ampules, weight percentage in 400 ml shampoo and weight percentage in 100 ml shampoo is disclosed in the following table.

| | | | % in | % in |
|---|---|---|---|---|
| | | 5 ml | shampoo | shampoo |
| Ingredients | % | ampules | 400 ml | 100 ml |
| Aloe vera extract-powder | 3 | 0.15 | 0.04 | 0.15 |
| Water | 41.2 | 8.24 | 2.06 | 8.24 |
| Horse tail tincture | 20 | 1 | 0.25 | 1 |
| Nettle tincture | 15 | 0.75 | 0.19 | 0.75 |
| Pea protein | 5 | 0.25 | 0.06 | 0.25 |
| Dandelion extract | 5 | 0.25 | 0.06 | 0.25 |
| Panthenol | 5 | 0.25 | 0.06 | 0.25 |
| Glycerin | 5 | 0.25 | 0.06 | 0.25 |
| Preservative 12 | 0.8 | 0.04 | 0.01 | 0.04 |

### Example—3

In an example, the hair care based additive composition for voluminous hair in weight percentage, grams per 5 ml ampules, weight percentage in 400 ml shampoo and weight percentage in 100 ml shampoo is disclosed in the following table.

| | | | % in | % in |
|---|---|---|---|---|
| | | 5 ml | shampoo | shampoo |
| Ingredients | % | ampules | 400 ml | 100 ml |
| Phytokeratin | 80 | 4 | 1 | 4 |
| Wheat Protein | 10 | 0.5 | 0.125 | 0.5 |
| (Kelyamin Liquid) | | | | |
| Pseudo Collagen | 5 | 0.25 | 0.0625 | 0.25 |
| Silk amino acids | 4.2 | 0.21 | 0.0525 | 0.21 |
| Preservative 12 | 0.8 | 0.04 | 0.01 | 0.04 |

### Example—4

In an example, the hair care based additive composition for dry brittle hair in weight percentage, grams per 5 ml ampules, weight percentage in 400 ml shampoo and weight percentage in 100 ml shampoo is disclosed in the following table.

| | | | % in | % in |
|---|---|---|---|---|
| | | 5 ml | shampoo | shampoo |
| Ingredients | % | ampules | 400 ml | 100 ml |
| Caprylic/Capric triglicerides | 70 | 3.5 | 0.875 | 3.5 |
| Prickly Pear Seed oil | 5 | 0.25 | 0.0625 | 0.25 |
| Bao bab oil | 10 | 0.5 | 0.125 | 0.5 |
| Oat oil | 10 | 0.5 | 0.125 | 0.5 |
| Argan oil | 4 | 0.2 | 0.05 | 0.2 |
| Vitamin E | 1 | 0.05 | 0.0125 | 0.05 |

### Example—5

in an example, the hair care based additive composition for dull hair in weight percentage, grams per 5 ml ampules, weight percentage in 400 ml shampoo and weight percentage in 100 ml shampoo is disclosed in the following table.

| | | | % in | % in |
|---|---|---|---|---|
| | | 5 ml | shampoo | shampoo |
| Ingredients | % | ampules | 400 ml | 400 ml |
| Caprylic/Capric triglicerides | 70 | 3.5 | 0.875 | 3.5 |
| Avocado oil | 5 | 0.25 | 0.0625 | 0.25 |
| Jojoba oil | 14 | 0.7 | 0.175 | 0.7 |
| Camellia seed oil | 10 | 0.5 | 0.125 | 0.5 |
| Castor oil | 2 | 0.1 | 0.025 | 0.1 |
| Oryza sativa oil | 2 | 0.1 | 0.025 | 0.1 |
| Safflower Oil - Carthamus | 2 | 0.1 | 0.025 | 0.1 |
| tinctorius | | | | |
| Vitamin E | 1 | 0.05 | 0.0125 | 0.05 |

### Example—6

In an example, the hair care based additive composition for frizzy hair in weight percentage, grams per 5 ml ampules, weight percentage in 400 ml shampoo and weight percentage in 100 ml shampoo is disclosed in the following table.

| | | | % in | % in |
|---|---|---|---|---|
| | | 5 ml | shampoo | shampoo |
| Ingredients | % | ampules | 400 ml | 100 ml |
| Water | 43.2 | 2.16 | 0.54 | 2.16 |
| Glycerin | 6 | 0.3 | 0.075 | 0.3 |
| Pantenol | 5 | 0.25 | 0.0625 | 0.25 |
| Phytokeratin | 15 | 0.75 | 0.19 | 0.75 |
| Brocoli oil | 5 | 0.25 | 0.0625 | 0.25 |
| Polyquaternium -7 | 5 | 0.25 | 0.0625 | 0.25 |
| Peg 40 hydrogenated castor oil | 10 | 0.5 | 0.125 | 0.5 |
| Preservative 12 | 0.8 | 0.04 | 0.01 | 0.04 |

### Example—7

In an example, the hair care based additive composition for dandruff or psoriasis treatment in weight percentage, grams per 5 ml ampules, weight percentage in 400 ml shampoo and weight percentage in 100 ml shampoo is disclosed in the following table.

| | | | % in | % in |
|---|---|---|---|---|
| | | 5 ml | shampoo | shampoo |
| Ingredients | % | ampules | 400 ml | 100 ml |
| Salycilyc acid | 1.5 | 0.075 | 0.018 | 0.075 |
| Propylen glicol | 72.5 | 3.625 | 0.90625 | 3.625 |
| Bisabolol | 1 | 0.05 | 0.0125 | 0.05 |
| Tea tree oil | 10 | 0.5 | 0.125 | 0.5 |
| Resemary oil | 10 | 0.5 | 0.125 | 0.5 |
| Cosmetic Prebiotic (inulin, | 5 | 0.25 | 0.0625 | 0.25 |
| Aqua, Alpha-glucan | | | | |
| oligosaccharide) | | | | |

### Example—8

In an example, the hair care based additive composition for oily scalp in weight percentage, grams per 5 ml ampules, weight percentage in 400 ml shampoo and weight percentage in 100 ml shampoo is disclosed in the following table.

| | | | % in | % in |
|---|---|---|---|---|
| | | 5 ml | shampoo | shampoo |
| Ingredients | % | ampules | 400 ml | 100 ml |
| Rose floral water | 57.2 | 2.86 | 0.715 | 2.86 |
| Cosmetic Prebiotic (inulin, | 10 | 0.5 | 0.125 | 0.5 |
| Aqua, Alpha-glucan | | | | |
| oligosaccharide) | | | | |
| Alpine extract | 10 | 0.5 | 0.125 | 0.5 |
| Tea tree oil | 2 | 0.1 | 0.025 | 0.1 |
| Clary sage oil | 3 | 0.15 | 0.038 | 0.15 |
| Cedar wood oil | 2 | 0.1 | 0.025 | 0.1 |
| PEG hydrogenated castor oil | 15 | 0.75 | 0.19 | 0.75 |
| Preservative 12 | 0.8 | 0.04 | 0.01 | 0.04 |

### Example—9

In an example, the hair care based additive composition for replumping hair filler in weight percentage, grams per 5 ml ampules, weight percentage in 400 ml shampoo and weight percentage in 100 ml shampoo is disclosed in the following table.

| | | | % in | % in |
|---|---|---|---|---|
| | | 5 ml | shampoo | shampoo |
| Ingredients | % | ampules | 400 ml | 100 ml |
| Phytokeratin | 80 | 4 | 1 | 4 |
| Panthenol | 10 | 0.5 | 0.125 | 0.5 |
| Pseudo Collagen | 10 | 0.5 | 0.125 | 0.5 |
| Preservative 12 | 0.8 | 0.04 | 0.01 | 0.04 |

Although a single embodiment of the invention has been illustrated in the accompanying drawings and described in the above detailed description, it will be understood that the invention is not limited to the embodiment developed herein, but is capable of numerous rearrangements, modifications, substitutions of parts and elements without departing from the spirit and scope of the invention.

The foregoing description comprises illustrative embodiments of the present invention. Having thus described exemplary embodiments of the present invention, it should be noted by those skilled in the art that the within disclosures are exemplary only, and that various other alternatives, adaptations, and modifications may be made within the scope of the present invention. Merely listing or numbering the steps of a method in a certain order does not constitute any limitation on the order of the steps of that method. Many modifications and other embodiments of the invention will come to mind to one skilled in the art to which this invention pertains having the benefit of the teachings presented in the foregoing descriptions. Although specific terms may be employed herein, they are used only in generic and descriptive sense and not for purposes of limitation. Accordingly, the present invention is not limited to the specific embodiments illustrated herein.

What is claimed is:

1. A composition for preparing a customized personal care product, comprising:
a base composition and at least one additive compositions;
wherein the base composition is at least one or combination of organic ingredients, organic origin ingredients and inorganic ingredients, and the additive compositions include at least one hair care based additive compositions.

2. The composition of claim 1, wherein the base composition includes sodium coco-sulphate, cocamidopropyl betaine, decyl glucoside, glycerin, caprylyl/capryl glucoside, lauryl glucoside, aloe barbadensis leaf juice powder, glyceryl oleate, coco-glucoside, citric acid, levulinic acid, sodium levulinate, sodium phytate, sodium benzoate, and water.

3. The composition of claim 1, is at least anyone of a health care product composition, beauty product composition, face serum product composition, body lotion product composition, and hair care product composition.

4. The composition of claim 1, wherein the hair care based additive composition for hair loss include,
(a) 40% of Wu Zhu extract (propanediol and Evodia rutaecarpa fruit extract),
(b) 5% of panthenol,
(c) 1% of caffeine,
(d) 40% of green tea extract,
(e) 0.5% of lactic acid,
(f) 2% of vitamin E,
(g) 1% of tea tree oil,
(h) 1% of rosemary oil,
(i) 0.5% of sodium benzoate, and
(j) 9% of PEG-40 hydrogenated castor oil.

5. The composition of claim 1, wherein the hair care based additive composition for hair root strengthening include,
(a) 3% of powdered Aloe vera extract,
(b) 41.2% of water,
(c) 20% of horse tail tincture,
(d) 15% of Nettle tincture,
(e) 5% of Pea protein,
(f) 5% of Dandelion extract,
(g) 5% of Panthenol,
(h) 5% of Glycerin, and
(i) 0.8% of Preservative 12 (Phenoxyethanol, Ethylhexylglycerin).

6. The composition of claim 1, wherein the hair care based additive composition for voluminous hair include,
(a) 80% of Phytokeratin,
(b) 10% of Wheat Protein,
(c) 5% of Pseudo Collagen,
(d) 4.2% of Silk amino acids, and
(e) 0.8% of Preservative 12 (Phenoxyethanol, Ethylhexylglycerin).

7. The composition of claim 1, wherein the hair care based additive composition for dry brittle hair include,
(a) 70% of Caprylic/Capric triglicerides,
(b) 5% of Prickly Pear Seed oil,
(c) 10% of Bao bab oil,
(d) 10% of Oat oil,
(e) 4% of Argan oil and
(f) 1% of Vitamin E.

8. The composition of claim 1, wherein the hair care based additive composition for dull hair include,
(a) 70% of Caprylic/Capric triglicerides,
(b) 5% of Avocado oil,
(c) 14% of Jojoba oil,
(d) 10% of Camellia seed oil,
(e) 2% of Castor oil,
(f) 2% of Rice bran oil,
(g) 2% of Safflower Oil, and
(h) 1% of Vitamin E.

9. The composition of claim 1, wherein the hair care based additive composition for frizzy hair include,
(a) 43.2% of water,
(b) 6% of Glycerin,
(c) 5% of Pantenol,
(d) 15% of Phytokeratin,
(e) 5% of Broccoli oil,
(f) 5% of Polyquaternium-7,
(g) 10% of PEG-40 hydrogenated castor oil, and
(h) 0.8% of Preservative 12 (Phenoxyethanol, Ethylhexylglycerin).

10. The composition of claim 1, wherein the hair care based additive composition for dandruff or psoriasis treatment include,
(a) 1.5% of Salicylic acid,
(b) 72.5% of Propylen glycol,
(c) 1% of Bisabolol,
(d) 10% of Tea tree oil,
(e) 10% of rosemary oil, and
(d) 5% of cosmetic prebiotic (inulin, Aqua, Alpha-glucan oligosaccharide).

11. The composition of claim 1, wherein the hair care based additive composition for oily scalp include,
(a) 57.2% of Rose floral water,
(b) 10% of Cosmetic Prebiotic (inulin, Aqua, Alpha-glucan oligosaccharide),
(c) 10% of Alpine extract,
(d) 2% of Tea tree oil,
(e) 3% of Clary sage oil,
(f) 2% of Cedar wood oil,
(g) 15% of PEG hydrogenated castor oil, and
(h) 0.8% of Preservative 12 (Phenoxyethanol, Ethylhexylglycerin).

12. The composition of claim 1, wherein the hair care based additive composition for replumping hair filler include,
(a) 80% of Phytokeratin,
(b) 10% of Panthenol,
(c) 10% of Pseudo Collagen, and
(d) 10% of Preservative 12 (Phenoxyethanol, Ethylhexylglycerin).

13. A method for preparing a customized personal care composition, comprising the step of:
(a) Adding at least one customized additive of predetermined composition from an ampoule in a base composition, and
(b) Mixing the base composition and at least one customized additive compositions to form a customized personal care composition,
wherein the base composition is at least one or combination of organic ingredients, organic origin ingredients and inorganic ingredients, and the additive compositions include at least one hair care based additive compositions.

14. The method of claim 13, wherein the base composition includes sodium coco-sulphate, cocamidopropyl betaine, decyl glucoside, glycerin, caprylyl/capryl glucoside, lauryl glucoside, aloe barbadensis leaf juice powder, glyceryl oleate, coco-glucoside, citric acid, levulinic acid, sodium levulinate, sodium phytate, sodium benzoate and water.

15. The method of claim 13, wherein the hair care based additive composition for hair loss include,
(a) 40% of Wu Zhu extract (propanediol and Evodia rutaecarpa fruit extract),
(b) 5% of panthenol,
(c) 1% of caffeine,
(d) 40% of green tea extract,
(e) 0.5% of lactic acid,
(f) 2% of vitamin E,
(g) 1% of tea tree oil,
(h) 1% of rosemary oil,
(i) 0.5% of sodium benzoate, and
(j) 9% of PEG-40 hydrogenated castor oil.

16. The method of claim 13, wherein the hair care based additive composition for hair root strengthening include,
(a) 3% of powdered Aloe vera extract,
(b) 41.2% of water,
(c) 20% of horse tail tincture,
(d) 15% of Nettle tincture,
(e) 5% of Pea protein,
(f) 5% of Dandelion extract,
(g) 5% of Panthenol,
(h) 5% of Glycerin, and
(i) 0.8% of Preservative 12 (Phenoxyethanol, Ethylhexylglycerin).

17. The method of claim 13, wherein the hair care based additive composition for voluminous hair include,
(a) 80% of Phytokeratin,
(b) 10% of Wheat Protein,
(c) 5% of Pseudo Collagen,
(d) 4.2% of Silk amino acids, and
(e) 0.8% of Preservative 12 (Phenoxyethanol, Ethylhexylglycerin).

18. The method of claim 13, wherein the hair care based additive composition for dry brittle hair include,
(a) 70% of Caprylic/Capric triglicerides,
(b) 5% of Prickly Pear Seed oil,
(c) 10% of Bao bab oil,
(d) 10% of Oat oil,
(e) 4% of Argan oil and
(f) 1% of Vitamin E.

19. The method of claim 13, wherein the hair care based additive composition for dull hair include,
(a) 70% of Caprylic/Capric triglicerides,
(b) 5% of Avocado oil,
(c) 14% of Jojoba oil,
(d) 10% of Camellia seed oil,
(e) 2% of Castor oil,
(f) 2% of Rice bran oil,
(g) 2% of Safflower Oil, and
(h) 1% of Vitamin E.

20. The method of claim 13, wherein the hair care based additive composition for frizzy hair include,
(a) 43.2% of water,
(b) 6% of Glycerin,
(c) 5% of Pantenol,
(d) 15% of Phytokeratin,
(e) 5% of Broccoli oil,
(f) 5% of Polyquaternium-7,
(g) 10% of PEG-40 hydrogenated castor oil, and
(h) 0.8% of Preservative 12 (Phenoxyethanol, Ethylhexylglycerin).

21. The method of claim 13, wherein the hair care based additive composition for dandruff or psoriasis treatment include,
(a) 1.5% of Salicylic acid,
(b) 72.5% of Propylen glycol,
(c) 1% of Bisabolol,
(d) 10% of Tea tree oil,
(e) 10% of rosemary oil, and
(d) 5% of cosmetic prebiotic (inulin, Aqua, Alpha-glucan oligosaccharide).

22. The method of claim 13, wherein the hair care based additive composition for oily scalp include,
(a) 57.2% of Rose floral water,
(b) 10% of Cosmetic Prebiotic (inulin, Aqua, Alpha-glucan oligosaccharide),
(c) 10% of Alpine extract,
(d) 2% of Tea tree oil,
(e) 3% of Clary sage oil,
(f) 2% of Cedar wood oil,
(g) 15% of PEG hydrogenated castor oil, and
(h) 0.8% of Preservative 12 (Phenoxyethanol, Ethylhexylglycerin).

23. The method of claim 13, wherein the hair care based additive composition for replumping hair filler include,
(a) 80% of Phytokeratin,
(b) 10% of Panthenol,
(c) 10% of Pseudo Collagen, and
(d) 10% of Preservative 12 (Phenoxyethanol, Ethylhexylglycerin).
